(19) United States
(12) Patent Application Publication
(10) Pub. No.: US 2022/0110512 A1
(43) Pub. Date: Apr. 14, 2022

(54) SYSTEMS AND METHODS FOR MITIGATING FOGGING IN ENDOSCOPIC IMAGING

(71) Applicant: Stryker Corporation, Kalamazoo, MI (US)

(72) Inventors: Chien Mien PANG, San Jose, CA (US); Eric Charles HUYNH, San Ramon, CA (US); Ajay RAMESH, Pleasanton, CA (US); Levey TRAN, Denver, CO (US); William H. L. CHANG, Milpitas, CA (US); Andrew Morgan ROBINSON, Coquitlam (CA)

(73) Assignee: Stryker Corporation, Kalamazoo, MI (US)

(21) Appl. No.: 17/497,876

(22) Filed: Oct. 8, 2021

Related U.S. Application Data
(60) Provisional application No. 63/090,107, filed on Oct. 9, 2020.

(51) Int. Cl. A61B 1/12; A61B 1/06; A61B 1/04

(52) U.S. Cl. CPC A61B 1/127; A61B 1/043; A61B 1/0638

(57) ABSTRACT

A method for defogging an optical component of an endoscope includes operating an illuminator in an imaging mode in which the illuminator generates illumination light for endoscopic imaging of a target, wherein at least a portion of the illumination light is generated by a light source that generates light having a first waveband at an imaging intensity level; and changing the operating mode of the illuminator from the imaging mode to a defogging mode in which an intensity level of the light having the first waveband is increased from the imaging intensity level to a defogging intensity level to warm and defog the optical component of the endoscope.

### CROSS-REFERENCE TO RELATED APPLICATIONS

This application claims the benefit of U.S. Provisional Application No. 63/090,107, filed Oct. 9, 2020, the entire contents of which are hereby incorporated by reference herein.

### FIELD

This disclosure relates generally to endoscopic imaging, and more specifically, to defogging an endoscope. Generally, the disclosure relates to the functioning of an endoscope.

### BACKGROUND

Minimally invasive surgery generally involves the use of a high-definition camera coupled to an endoscope inserted into a patient to provide a surgeon with a clear and precise view within the body. The endoscope emits light from its distal end to illuminate the surgical cavity and receives light reflected or emitted by tissue within the surgical cavity through a lens or window located at the distal end of the endoscope. A long-standing challenge associated with endoscopic imaging is fogging of the distal lens or window. Surgical rooms are generally kept at a dry temperature between 20° C. and 24° C. This contrasts sharply with the environment within a surgical cavity, which is generally above 37° C. and more than 85% relative humidity. The temperature of the relatively cold endoscope when first inserted into the surgical cavity brings moisture surrounding the endoscope in the surgical cavity to its dew point, causing the accumulation of condensation on the distal lens or window. This condensation, or fogging, can obscure the endoscopic camera's field of view. Endoscope fogging can also be caused by changes in the environment of the surgical cavity during a procedure, such as from cauterization of tissue, which produces alterations in heat and moisture.

Surgeons preferring not to interrupt surgery when endoscope fogging occurs may simply continue with foggy images, sacrificing image quality and the ability to see details through the endoscope. Surgeons requiring clear images may wait until fogging clears on its own when the endoscope temperature equalizes with that of the surgical cavity, which may take a long while, or may interrupt the surgery to clear the fogging, such as by removing the endoscope from the surgical cavity and wiping the distal lens or window clean. However, these techniques significantly interrupt the surgery and, in the case of removal and reinsertion, may ultimately prove ineffective since the endoscope may fog again when reinserted into the surgical cavity. Alternatively, surgeons may attempt to clear fogging without removing the endoscope from the surgical cavity by wiping the endoscope against tissue within the surgical cavity, but this can make matters worse by smudging debris from the tissue onto the endoscope and requires the endoscope to be moved from its imaging position. Thus, many known techniques for dealing with endoscope fogging are disruptive at best and ineffective at worst.

### SUMMARY

According to an aspect, functioning of the endoscope is improved in that fogging of an optical component of an endoscope is cleared by increasing the level of illumination light provided through the endoscope to warm the endoscope to a temperature sufficient to clear the fogging while the endoscope pre-inserted in a pre-made surgical cavity remains in the surgical cavity. To clear fogging of the endoscope, the illuminator providing illumination light to the endoscope for imaging may enter a defogging mode in which the level of illumination provided to the endoscope is increased above a level provided during normal imaging. The level of illumination can be increased by activating additional light sources, by increasing the intensity of light provided by one or more activate light sources, or through a combination of these. While the additional light results primarily in increased illumination of the surgical field, a portion of the additional light energy is converted to heat energy that heats the distal portion of the endoscope. Once the endoscope reaches a temperature sufficient to clear the fogging, the illuminator can revert to providing a level of illumination suitable for imaging. Thus, fogging can be cleared from the endoscope quickly and effectively, without requiring any movement of the endoscope, much less removal from the surgical cavity. The fogging may be cleared from one or more optical components of the endoscope, such as the distal lens or window of the endoscope and/or the proximal lens or window of the endoscope.

According to an aspect, an endoscope can be pre-warmed prior to insertion into a surgical cavity to prevent fogging of the endoscope once inserted into the surgical cavity. The endoscope can be pre-warmed using illumination light, alone, or in combination with a warming cap. The warming cap can be configured to slide over the distal portion of the endoscope so that the distal portion of the endoscope is covered by the warming cap. The warming cap may be configured to reflect light emitted from the endoscope back onto the endoscope and to insulate the distal portion of the endoscope, resulting in faster heating of the endoscope.

According to an aspect, an endoscope defogging procedure is automatically executed in response to detection of fogging via image processing. To detect defogging, one or more feature vectors can be calculated for images generated by the endoscopic camera. These feature vectors can be analyzed to predict whether the images include fogginess. For example, the feature vectors can be analyzed by a Machine Learning algorithm trained on foggy and non-foggy endoscopic images. Upon detecting fogginess in one or more images, a defogging procedure can be automatically executed.

According to an aspect, the distal lens or window of the endoscope, or another optical component of the endoscope can be configured to mitigate fogging. For example, the distal lens or window or other optical component can have etched nano-structures that have hydrophobic properties to discourage water from condensing on the lens or hydrophilic properties to cause condensation to form a layer, rather than droplets. In some variations, a thermochromic layer is applied to the distal lens or window or other optical component to increase the heating of the distal lens or window or other optical component from illumination light provided through the endoscope.

According to an aspect, a method for defogging an optical component of an endoscope includes operating an illuminator in an imaging mode in which the illuminator generates illumination light for endoscopic imaging of a target, wherein at least a portion of the illumination light is generated by a light source that generates light having a first waveband at an imaging intensity level; and changing the operating mode of the illuminator from the imaging mode to a defogging mode in which an intensity level of the light having the first waveband is increased from the imaging intensity level to a defogging intensity level to warm and defog the optical component of the endoscope.

Optionally, the method may further include changing the mode of the illuminator back to the imaging mode after the optical component of the endoscope is at least partially defogged. Optionally, the mode of the illuminator is changed back to the imaging mode after a predefined period of time in the defogging mode.

Optionally, the illuminator comprises a plurality of light sources, each light source of the plurality of light sources generates a different waveband of light, and increasing the intensity level of the illumination light having the first waveband comprises increasing power provided to at least the light source that generates the light having the first waveband from an imaging power level to a defogging power level. Optionally, the light source that generates the light having the first waveband comprises a plurality of light emitters that each emit light having the first waveband.

Optionally, the first waveband is a waveband in the visible light spectrum.

Optionally, the first waveband is a near infrared waveband for exciting a fluorescence target.

Optionally, the first waveband is an ultraviolet waveband.

Optionally, a second portion of the illumination light is generated by a second light source generating light having a second waveband, and an intensity level of the light having the second waveband provided by the second light source is increased in the defogging mode relative to the imaging mode.

Optionally, the mode of the illuminator is changed from the imaging mode to the defogging mode in response to a user input.

Optionally, the method may further include receiving image data generated by an endoscopic camera connected to the endoscope while the illuminator is in the imaging mode; analyzing the image data to automatically detect fogging of the optical component of the endoscope; and in response to automatically detecting fogging of the optical component, sending a signal to the illuminator that changes the operating mode of the illuminator from the imaging mode to the defogging mode. Optionally, the method may further include sending a subsequent signal to the illuminator to change the operating mode of the illuminator back to the imaging mode. Optionally, the method may further include receiving additional image data while the illuminator is in the defogging mode; analyzing the image data to automatically detect that the optical component of the endoscope has been defogged; and in response to automatically detecting that the optical component of the endoscope has been defogged, sending a signal to the illuminator to change the operating mode of the illuminator back to the imaging mode.

Optionally, the optical component comprises a distal lens or window at the distal end of the endoscope.

Optionally, the optical component comprises a proximal lens or window at the proximal end of the endoscope.

Optionally, the endoscope comprises a plurality of optical fibers for carrying the illumination light and at least a portion of the plurality of optical fibers are located radially outwardly of the optical component. Optionally, at least a second portion of the plurality of optical fibers direct light onto the optical component. Optionally, an outer surface of the optical component and termination surfaces of at least the portion of the plurality of optical fibers that are located radially outwardly of the optical component are coplanar.

Optionally, the illuminator is configured to operate in a plurality of defogging modes for endoscopes of different types.

Optionally, the method further includes automatically determining a type of the endoscope, wherein the defogging intensity level in the defogging mode is based at least partially on the determined type of the endoscope. Optionally, the mode of the illuminator is changed back to the imaging mode after a predefined period of time in the defogging mode, and wherein the predefined period of time is based on the type of the endoscope.

Optionally, the method further includes automatically detecting the absence of an endoscope and, while the absence is detected, disabling the defogging mode.

Optionally, the optical component is configured to transmit the light having a first waveband.

Optionally, the endoscope has been located in an existing surgical cavity prior to defogging of the optical component of the endoscope.

According to an aspect, a system comprising one or more processors configured for: operating an illuminator in an imaging mode in which the illuminator generates illumination light for endoscopic imaging of a target with an endoscope comprising an optical component, wherein at least a portion of the illumination light is generated by a light source that generates light having a first waveband at an imaging intensity level; and changing the operating mode of the illuminator from the imaging mode to a defogging mode in which an intensity level of the light having the first waveband is increased from the imaging intensity level to a defogging intensity level to warm and defog the optical component of the endoscope.

Optionally, the optical component comprises a proximal lens or window at the proximal end of the endoscope.

Optionally, the optical component comprises a distal lens or window at the distal end of the endoscope.

Optionally, the system is communicatively connected to the illuminator and the system is configured to send a signal to the illuminator to change the operating mode of the illuminator from the imaging mode to the defogging mode.

Optionally, the signal comprises information corresponding to the defogging intensity level.

Optionally, the one or more processors are configured for analyzing image data received from an endoscopic imager to detect fogging of the optical component and changing the operating mode of the illuminator from the imaging mode to the defogging mode in response to detecting fogging of the optical component.

Optionally, the one or more processors are further configured for changing the mode of the illuminator back to the imaging mode after the optical component of the endoscope is at least partially defogged. Optionally, the one or more processors are further configured for changing the mode of the illuminator back to the imaging mode after a predefined period of time in the defogging mode.

Optionally, the system comprises the illuminator and the illuminator comprises a plurality of light sources, each light source of the plurality of light sources generates a different waveband of light, and increasing the intensity of the illumination light having the first waveband comprises increasing power provided to at least the light source that generates the light having the first waveband from an imaging power level to a defogging power level.

Optionally, the light source that generates the light having the first waveband comprises a plurality of light emitters that each emit light having the first waveband.

Optionally, the first waveband is a waveband in the visible light spectrum.

Optionally, the first waveband is a near infrared waveband for exciting a fluorescence target.

Optionally, a second portion of the illumination light is generated by a second light source generating light having a second waveband, and an intensity level of the light having the second waveband provided by the second light source is increased in the defogging mode relative to the imaging mode.

Optionally, the one or more processors are configured to change the operating mode of the illuminator from the imaging mode to a defogging mode in response to a user input.

Optionally, the one or more processors are further configured for: receiving image data generated by an endoscopic camera connected to the endoscope while the illuminator is in the imaging mode; analyzing the image data to automatically detect fogging of the optical component of the endoscope; and in response to automatically detecting fogging of the optical component, sending a signal to the illuminator that changes the operating mode of the illuminator from the imaging mode to the defogging mode.

Optionally, the one or more processors are further configured to send a subsequent signal to the illuminator to change the operating mode of the illuminator back to the imaging mode. Optionally, the one or more processors are further configured for: receiving additional image data while the illuminator is in the defogging mode; analyzing the image data to automatically detect that the optical component of the endoscope has been defogged; and in response to automatically detecting that the optical component of the endoscope has been defogged, sending a signal to the illuminator to change the operating mode of the illuminator back to the imaging mode.

Optionally, the system comprises the endoscope and the endoscope comprises a plurality of optical fibers for carrying the illumination light and at least a portion of the plurality of optical fibers are located radially outwardly of the optical component. Optionally, at least a second portion of the plurality of optical fibers direct light onto the optical component. Optionally, an outer surface of the optical component and termination surfaces of at least the portion of the plurality of optical fibers that are located radially outwardly of the optical component are coplanar.

Optionally, the one or more processors are further configured for automatically detecting the absence of an endoscope and, while the absence is detected, disabling the defogging mode.

Optionally, the system comprises the illuminator and the illuminator is configured to operate in a plurality of defogging modes for endoscopes of different types.

Optionally, the one or more processors are further configured for automatically determining a type of the endoscope, wherein the defogging intensity level in the defogging mode is based at least partially on the determined type of the endoscope.

Optionally, the one or more processors are configured to change the mode of the illuminator back to the imaging mode after a predefined period of time in the defogging mode, and wherein the predefined period of time is based on the type of the endoscope.

Optionally, the system comprises the endoscope and the optical component is configured to transmit the light having a first waveband.

Optionally, the one or more processors are configured to change the operating mode of the illuminator to the defogging mode while the endoscope is in use, such as after the endoscope has been located in a pre-made surgical cavity.

According to an aspect, a warming cap configured for mounting on a distal portion of an endoscope to warm the distal portion of the endoscope via light energy provided by the endoscope includes: a sleeve for removably positioning on the distal portion of the endoscope, the sleeve comprising a closed end portion that is adjacent an optical component of the endoscope when the warming cap is mounted on the endoscope, the closed end portion configured to reflect at least a portion of light emitted from the distal portion of the endoscope onto the distal portion of the endoscope to heat the distal portion of the endoscope; and an insulating portion at least partially surrounding the sleeve for thermally insulating the sleeve.

Optionally, the warming cap further includes a resilient retainer for resiliently positioning against an outer surface of the distal portion of the endoscope to retain the warming cap on the endoscope. Optionally, the resilient retainer is an o-ring.

Optionally, the sleeve is formed of a metal and the insulating portion is formed of a polymeric material.

Optionally, the warming cap is passive.

Optionally, the warming cap is sterilizable as a unit.

Optionally, the sleeve is configured so that the endoscope bottoms out on the closed end portion of the sleeve when fully inserted into the sleeve.

According to an aspect, a method includes mounting a warming cap on a distal portion of an endoscope so that the warming cap covers a distal end of the endoscope; providing light from an illuminator to the endoscope so that the light is emitted from the distal end of the endoscope; and warming the distal portion of the endoscope with the warming cap solely via the light emitted from the distal end of the endoscope.

Optionally, the method includes warming the distal portion of the endoscope with the warming cap for a predetermined period of time.

Optionally, the method includes dismounting the warming cap prior to inserting the endoscope into the surgical cavity for endoscopic imaging.

Optionally, providing light from an illuminator to the endoscope comprises providing an intensity of light that is elevated relative to an intensity of light provided during endoscopic imaging.

Optionally, the light provided to the illuminator comprises visible light.

Optionally, the light provided to the illuminator comprises near infrared light.

According to an aspect, a method for automatically detecting fogging of an endoscope includes receiving image data corresponding to an image captured by an endoscopic imager via the endoscope; computing at least one feature vector for at least one region of interest of the image from the image data; and analyzing the at least one feature vector to automatically detect fogging of the endoscope. The endoscope can have been inserted in a surgical cavity prior to starting the method for automatically detecting fogging.

Optionally, the at least one feature vector comprises at least one of a dispersion index, a gradient magnitude, and FFT energy.

Optionally, fogging of the endoscope is detected for each of a plurality of regions of the image.

Optionally, the method includes initiating a defogging operation based on detecting fogging of the endoscope. Optionally, the defogging operation is initiated based on detecting fogging in a plurality of regions of interest of the image. Optionally, the defogging operation is initiated based on detecting fogging in a predetermined percentage of the plurality of regions of interest of the image. Optionally, the defogging operation is initiated based on detecting fogging in at least one previously captured image. Optionally, the method includes computing and analyzing the at least one feature vector for images received during the defogging operation and ceasing the defogging operation based on failing to detect fogging of the endoscope in at least a portion of at least one image received during the defogging operation.

Optionally, analyzing the at least one feature vector comprises analyzing the at least one feature vector using a machine learned model trained on labeled images.

Optionally, the at least one feature vector is analyzed using a classifier.

Optionally, computing the at least one feature vector comprises computing a dispersion index map, at least in part, by dividing the region of interest into sub-regions and for each sub-region: computing a median pixel value for the respective sub-region; computing a distance for each pixel in the respective sub-region from the median pixel value for the respective sub-region; and computing an average distance for the sub-region, wherein the at least one feature vector comprises the average distance for each sub-region. Optionally, the region of interest is divided into sub-regions via a sliding window technique.

Optionally, the image data is received intraoperatively.

Optionally, the image data comprises an image of a surgical cavity.

According to an aspect, a computing system includes one or more processors configured for: receiving image data corresponding to an image captured by an endoscopic imager; computing at least one feature vector for at least one region of interest of the image from the image data; and analyzing the at least one feature vector to detect fogging of the endoscope.

Optionally, the computing system comprises a controller configured to transmit a signal to at least one communicatively connected component to initiate a defogging operation based on detecting fogging of the endoscope.

Optionally, the system comprises the at least one communicatively connected component.

Optionally, the at least one communicatively connected component comprises at least one of an insufflator and an illuminator.

Optionally, the computing system is configured to receive the image data from an endoscopic camera controller.

Optionally, the computing system comprises an endoscopic camera controller.

Optionally, the at least one feature vector comprises at least one of a dispersion index, a gradient magnitude, and FFT energy.

Optionally, the one or more processors are configured for detecting fogging of the endoscope for each of a plurality of regions of the image.

Optionally, the one or more processors are further configured for initiating a defogging operation based on detecting fogging of the endoscope. Optionally, the defogging operation is initiated based on detecting fogging in a plurality of regions of interest of the image. Optionally, the one or more processors are configured for initiating the defogging operation based on detecting fogging in a predetermined percentage of the plurality of regions of interest of the image. Optionally, the one or more processors are configured for initiating the defogging operation based on detecting fogging in at least one previously captured image. Optionally, the one or more processors are configured for computing and analyzing the at least one feature vector for images received during the defogging operation and ceasing the defogging operation based on failing to detect fogging of the endoscope in at least a portion of at least one image received during the defogging operation.

Optionally, the one or more processors are configured for analyzing the at least one feature vector using a machine learned model trained on labeled images.

Optionally, the one or more processors are configured for analyzing the at least one feature vector using a classifier.

Optionally, the one or more processors are configured for computing the at least one feature vector by computing a dispersion index map, at least in part, by dividing the region of interest into sub-regions and for each sub-region: computing a median pixel value for the respective sub-region; computing a distance for each pixel in the respective sub-region from the median pixel value for the respective sub-region; and computing an average distance for the sub-region, wherein the at least one feature vector comprises the average distance for each sub-region. Optionally, the region of interest is divided into sub-regions via a sliding window technique.

Optionally, the one or more processors is configured to receive the image data intraoperatively.

According to an aspect, an endoscope for an endoscopic imaging system includes a tube for insertion into a surgical cavity and an optical component for receiving light from the surgical cavity during endoscopic imaging, wherein the optical component comprises an etched nano-structure array configured to retard formation of water condensation droplets on the optical component when the endoscope is positioned in the surgical cavity during endoscopic imaging.

Optionally, the etched nano-structure is hydrophobic to retard condensing of water on the optical component.

Optionally, the etched nano-structure is hydrophilic to retard the formation of water droplets from condensation on the optical component.

According to an aspect, an endoscope for an endoscopic imaging system includes a tube for insertion into a surgical cavity and an optical component for receiving light from the surgical cavity during endoscopic imaging, wherein the optical component comprises a thermochromic layer configured to generate heat in response to receiving light to heat the optical component to prevent fogging.

According to an aspect, an endoscope for an endoscopic imaging system includes a tube for insertion into a surgical cavity and an optical component located at a distal end of the tube for transmitting illumination light to the surgical cavity during endoscopic imaging, wherein the optical component comprises a thermochromic layer configured to generate heat in response to receiving light to heat the optical component to prevent fogging.

According to an aspect, a non-transitory computer readable storage medium stores one or more programs for execution by one or more processors of an endoscopic imaging system, and the one or more programs include instructions for performing any of the methods described above.

It will be appreciated that any of the aspects, features and options described in view of any the systems described above apply equally to the corresponding methods and computer-readable storage mediums, and vice versa. It will also be clear that any one or more of the characteristics of any one or more of the systems, methods, and/or computer-readable storage mediums recited above may be combined, in whole or in part, with one another and/or with any other features or characteristics described elsewhere herein.

It will be appreciated that the methods described herein relate to the functioning of the endoscope as such. There is no functional relationship between the defogging of the endoscope as such and any therapeutic and/or surgical objectives of a medical practitioner using such endoscope. In particular, controlling the defogging functioning is independent of any acts of inserting an endoscope in a (pre-made) body cavity. Methods of defogging of an endoscope are described herein, wherein a step of inserting the endoscope into the body is excluded from such methods. In particular, methods of defogging of an endoscope are disclosed wherein the endoscope has been pre-inserted into the body.

### BRIEF DESCRIPTION OF THE FIGURES

The invention will now be described, by way of example only, with reference to the accompanying drawings, in which:

FIG. 1 illustrates an exemplary system for endoscope defogging using illumination energy;

FIGS. 2A and 2B are cross-sectional and front views, respectively, of a distal portion of an exemplary endoscope;

FIG. 3 is a block diagram illustrating components of an exemplary illuminator that may be used for endoscope defogging;

FIG. 4 illustrates an exemplary method for heating a distal end of an endoscope for defogging or preventing fogging of one or more optical components at the distal end of the endoscope;

FIG. 5A illustrates an example of a light spectrum generated by an exemplary illuminator operating in a white light imaging mode;

FIG. 5B illustrates an example of a light spectrum generated by an exemplary illuminator operating in a defogging mode;

FIG. 6 is a bar graph of exemplary illuminator light source drive currents illustrating two exemplary illuminator defogging modes;

FIG. 7 illustrates an exemplary method for endoscope defogging;

FIG. 8 is a flow diagram of an exemplary method for automatically detecting fogging on one or more optical components of an exemplary endoscope via image processing;

FIGS. 9A and 9B illustrate an exemplary non-foggy image and an exemplary foggy image, respectively; FIG. 9C is a plot of pixel values in RGB space for the foggy image of FIG. 9A and FIG. 9D is a plot of pixel values in RGB space for the non-foggy image of FIG. 9B;

FIG. 10 illustrates an exemplary method for calculating a dispersion index feature vector for an exemplary endoscopic image;

FIGS. 11A and 11B illustrate exemplary non-foggy and foggy images, respectively; FIGS. 11C and 11D illustrate the results of an exemplary dispersion index calculation for a region of interest in the non-foggy image of FIG. 11A and the foggy image of FIG. 11B, respectively; FIGS. 11E and 11F are exemplary heat maps of the gradient magnitude for the non-foggy image of FIG. 11A and the foggy image of FIG. 11B, respectively; FIGS. 11G and 11H are exemplary heat maps of the FFT energy for the non-foggy image of FIG. 11A and the foggy image of FIG. 11B, respectively;

FIG. 12 illustrates an exemplary warming cap for enhancing warming a distal end of an endoscope using the light transmitted by the endoscope;

FIG. 13 illustrates a distal portion of an exemplary endoscope in which an exemplary thermochromic layer is provided to enhance distal window heating;

FIG. 14 is a conceptual illustration of etching of nano-structures on a distal window of a distal portion of an exemplary endoscope;

FIG. 15 is a block diagram of an exemplary computing system; and

FIG. 16 illustrates a haze covering an entire display screen that can be cleared via defogging.

### DETAILED DESCRIPTION

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described herein are endoscopic imaging systems and methods for mitigating endoscope fogging for improved endoscopic imaging. According to various aspects, fogging of one or more optical components of an endoscope, such as the distal lens or window of the endoscope or the proximal lens or window of the endoscope, is cleared by increasing a level of illumination light transmitted through the endoscope to heat the optical component(s) to a temperature sufficient to clear the fogging. An illuminator that provides illumination light to the endoscope for illuminating tissue within a surgical cavity for imaging may switch from an imaging illumination mode in which the illuminator generates a level of light suitable for imaging to a defogging illumination mode in which the level of light is increased. A portion of the additional light energy is converted to heat at the distal end of the endoscope where the fiber optics carrying the light terminate, which raises the temperature of the optical component(s) at the distal end of the endoscope. Similarly, a portion of the additional light energy is converted to heat in the proximal region of the endoscope at the interface between the fiber optics of the endoscope and the light guide, which raises the temperature of the optical component(s) at the proximal end of the endoscope. The illuminator can remain in the defogging mode for a period of time sufficient to raise the temperature of the optical component(s), which causes the fogging on the optical component(s) to dissipate.

According to various aspects, the increased level of illumination light provided by the endoscope can be generated by activating one or more light sources of the endoscope and/or increasing a power to one or more light sources of the endoscope. For example, the level of light from one or more visible light sources may remain the same when switching to the defogging mode and an infrared light source used for fluorescence excitation and/or an ultraviolet light source can be additionally activated to provide a higher level of illumination to the endoscope over and above that provided during imaging and/or the level of light provided by the one or more visible light sources can increase.

According to various aspects, an image processing system is configured to automatically detect endoscope fogging via image processing during endoscopic imaging and automatically initiate a defogging procedure, such as by switching the illuminator to the defogging mode. The system may automatically detect fogging by calculating one or more feature vectors for an image and analyzing the feature vector(s), such as using a Machine Learning algorithm trained on foggy images, to predict whether fogging is present in the image. A defogging procedure may be triggered when a predefined proportion of an image is affected by fog and/or when a predefined proportion of a time series of images (e.g., video frames) includes fogginess.

By automatically detecting and clearing fog from the optical component of the endoscope, the surgical staff can continue performing surgery with minimal delay, avoiding the disruptions to the surgery experienced either waiting for the fogging to clear up as the endoscope warms up over time inside the surgical cavity or using conventional fog clearing techniques like removing the endoscope from the surgical cavity and warming it up before reinserting it into the surgical cavity.

Optionally, an endoscope can be pre-warmed before insertion into the surgical cavity to prevent or reduce the likelihood of fogging upon initial insertion of the endoscope into the surgical cavity. The endoscope may be pre-warmed using illumination light, alone, or in combination with a passive warming cap that is removably positioned over the distal end of the endoscope to increase the rate of heating of the endoscope by reflecting illumination light back onto the endoscope and trapping heat.

Optionally, a distal window or lens of an endoscope includes a thermochromic layer that is configured to enhance the illumination light heating effect by converting some incident light energy into heat. This can reduce the time required for defogging and can enable the distal window to maintain a sufficiently elevated temperature in the surgical cavity such that fogging cannot form.

Optionally, a distal window of the endoscope includes an etched nano-structure array configured to retard formation of fog related water droplets on the distal window while permitting imaging light to pass through. In some variations, the nano-structure array has hydrophobic properties that retard the condensing of water on the distal window. In other variations, the nano-structure array has hydrophilic properties that causes water condensation to spread across the distal window rather than forming droplets. This results in more even distribution of incident light, improving image quality relative to fog-related water droplets.

According to various aspects, the systems and methods described herein can not only help improve surgical outcomes by improving the quality of the surgical images generated during surgery, but can also decrease the overall time needed to perform a surgical procedure by eliminating the need for the surgical staff to defog the endoscope using cumbersome and lengthy procedures (often on multiple occasions during a surgery). The automatic detection of fogginess and automatic initiating of defogging procedures can reduce the cognitive load on the surgeon, who no longer needs to determine when images are foggy and no longer has to interrupt surgery to perform a manual defogging operation. This can improve surgical outcomes since fogging can occur during critical steps of a surgical procedure where a clear surgical image is important.

For purposes of this disclosure, the terms lens and window (as in “distal lens” and “distal window”) are used interchangeably. As such, for the purposes of this disclosure, the term window encompasses a lens and the term lens encompasses a window.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

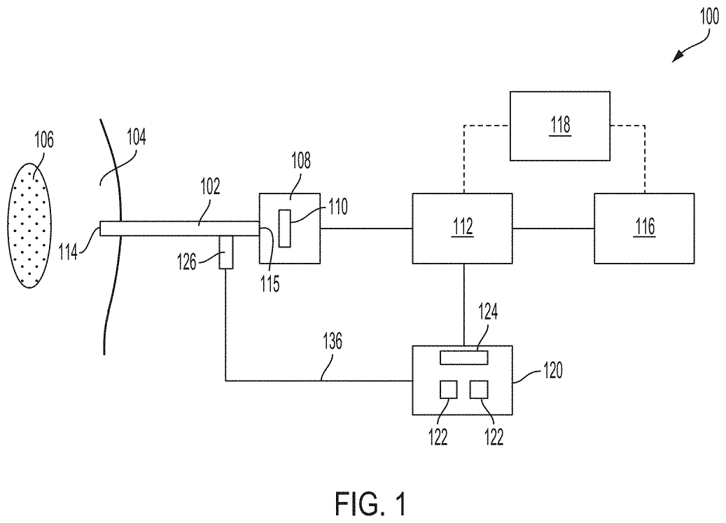

FIG. 1 illustrates an exemplary system 100 for endoscope defogging using illumination energy. System 100 includes an endoscope 102 suitable for insertion into a surgical cavity 104 for imaging tissue 106 within the surgical cavity 104 during a medical procedure and an illuminator 120 that provides illumination light to the endoscope 102, which emits the light from its distal end 114 for illuminating the tissue 106. As discussed further below, one or more optical components of the endoscope 102, such as a window or lens at the distal end 114 of the endoscope 102 and/or a window or lens at the proximal end 115, can be heated by energy from the illumination light provided by the illuminator 120 to defog or prevent fogging of the one or more optical components.

The endoscope 102 may extend from an endoscopic camera head 108 that includes one or more imaging sensors 110. As is well known in the art, light reflected and/or emitted (such as fluorescence light emitted by fluorescing targets that are excited by fluorescence excitation illumination light) from the tissue 106 is received by the distal end 114 of the endoscope 102. The light is propagated by the endoscope 102, such as via one or more optical components (for example, one or more lenses, prisms, light pipes, or other optical components), to the camera head 108, where it is directed onto the one or more imaging sensors 110. One or more filters (not shown) may be included in the endoscope 102 and/or camera head 108 for filtering a portion of the light received from the tissue 106 (such as fluorescence excitation light).

The one or more imaging sensors 110 generate pixel data that can be transmitted to a camera control unit 112 that is communicatively connected to the camera head 108. The camera control unit 112 generates one or more images from the pixel data. As used herein “images” encompasses single images and video frames. The images can be transmitted to an image processing unit 116 for further image processing, storage, display, and/or routing to an external device (not shown). The images can be transmitted to one or more displays 118, from the camera control unit 112 and/or the image processing unit 116, for visualization by medical personnel, such as by a surgeon for visualizing the surgical field 104 during a surgical procedure on a patient.

The illuminator 120 generates illumination light and provides the illumination light to the endoscope 102 via light guide 136, which may comprise, for example, one or more fiber optic cables, with the light guide 136 coupled to the endoscope 102 at the light post 126 in the proximal region of the endoscope. The illumination light is emitted from the distal end 114 of the endoscope 102 and illuminates the tissue 106. As used herein, “illumination light” refers to light that may be used to illuminate tissue for the purposes of imaging the tissue. At least a portion of the illumination light may be light having a waveband in the visible spectrum that is reflected by the tissue 106 and captured by the one or more imaging sensors 110 for generating visible light imaging data. Optionally, at least a portion of the illumination light can be fluorescence excitation light for exciting one or more fluorescing targets in the tissue, which can include one or more fluorescence agents and/or one or more auto-fluorescing targets. Light emitted by the one or more fluorescence targets may be captured by the one or more imaging sensors for generating fluorescence imaging data. The illumination light can include any desirable wavebands or combination of wavebands.

In this example, the illuminator 120 includes one or more light sources 122 that each generate one or more wavebands of light and a controller 124 for controlling the light sources 122. The light sources 122 generate illumination light for illuminating the tissue 106 via the endoscope 102. The controller 124 can be configured to activate and deactivate the individual light sources 122 and/or adjust a power level of the light sources 122 to adjust the level of intensity (i.e., the luminance) of light generated by the individual light sources 122. The controller 124 may control one or more light sources 122 based on one or more control signals received from camera control unit 112 and/or image processing unit 116. Control signals received from the camera control unit 112 and/or image processing unit 116 can instruct the controller 124 how to control individual light sources 122, such as by including instructions to activate or deactivate individual light sources 122 and/or to set one or more individual light sources 122 at a specified power/intensity level. For example, the camera control unit 112 and/or image processing unit 116 may determine that a specific color and/or intensity adjustment is needed based on analysis of pixel data generated by the endoscopic camera head 108 and the controller 124 may receive instructions specifying the adjustments. Optionally, the control signals correspond to an instruction to enter a predefined mode. For example, the controller 124 may receive an instruction to enter a white light mode or a defogging mode, and the controller 124 may respond by activating/deactivating individual light sources 122 and/or set individual light source power levels based on predefined configuration information.

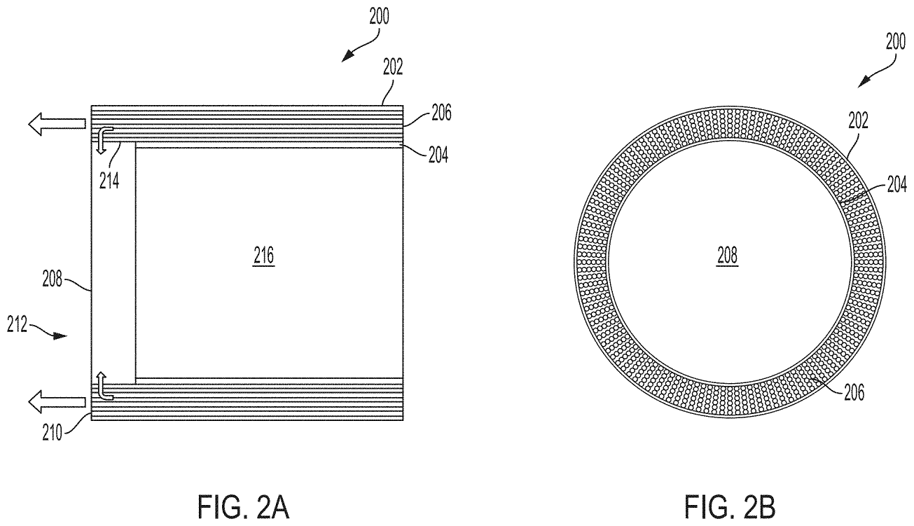

FIGS. 2A and 2B are cross-sectional and front views, respectively, of an exemplary distal portion of an endoscope 200 that can be used in system 100. Endoscope 200 includes an outer tube 202, and inner tube 204, and a plurality of fiber optics 206 arranged annularly between the inner tube 204 and outer tube 202. A distal window 208 (distal window and distal lens are used interchangeably herein) is located radially inwardly of the inner extent 214 of the plurality of fiber optics 206. The distal ends 210 of the plurality of fiber optics 206 may be coplanar with the outer (distal) surface of the distal window 208. At least a portion of the fiber optics may terminate at the distal window such that distal end surfaces of the portion of the fiber optics abut an inner surface of the distal window and light exiting the distal ends of the fiber optics passes through the distal window.

Illumination light generated by an illuminator, such as illuminator 120 of system 100 of FIG. 1, and provided to the endoscope 200 is carried along the plurality of fiber optics 206 from near the proximal end (not shown) of the endoscope 200 to the distal end 212 of the endoscope 200 where the light is emitted from the distal ends 210 of the plurality of fiber optics 206. Light from the tissue being imaged, which can include illumination light reflected from the tissue and fluorescence light emitted by the tissue, travels through the distal window 208 and through the bore 216 of the inner tube 204 (such as via one or more optical components) to one or more imaging sensors in the camera head.

Due to attenuation of the light by the plurality of fiber optics 206, a portion of the illumination light traveling through the fiber optics 206 is converted to heat during transmission of the light. This heat can warm the distal window 208, such as via conduction. However, due to improvements in camera technology, the relatively high sensitivity of the latest endoscopic cameras means that lower levels of illumination are required for imaging. As a result, the light loss at the distal end of the endoscope decreases, reducing the amount of heat generated by the illumination light at the distal end during normal imaging such that the temperature of the distal window 208 cannot be maintained above the dew point of the water vapor within the surgical cavity. This can lead to fogging of the distal window 208 while imaging during the endoscopic procedure. Accordingly, as discussed further below, the illuminator may enter a defogging mode in which an increased level of illumination light is provided to the endoscope. This increased level of light leads to more light energy being converted to heat at the distal end of the endoscope, which increases heating of the distal window. The defogging mode can continue until the temperature of the distal end of the endoscope is raised above the dew point within the surgical cavity such that any fog on the distal window dissipates.

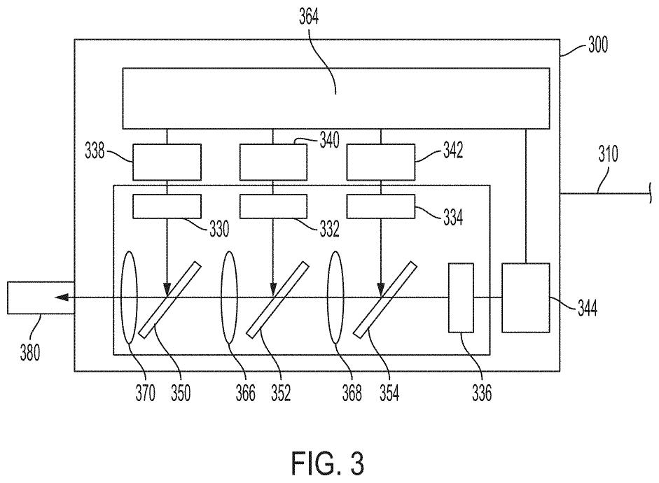

FIG. 3 is a block diagram illustrating components of an exemplary illuminator 300 that may be used as illuminator 120 of system 100. Illuminator 300 may be configured for generating white light as well as fluorescence excitation light, such as infrared light. The illuminator 300 includes four light sources—a laser diode 330, a first LED 332, a second LED 334, and a third LED 336. The laser diode 330 may be configured to generate fluorescence excitation light and the three LEDs may be configured to generate visible light, such as red, green, and blue light (e.g., to provide white light). The laser diode 330 is activated by a laser diode driver 338. The first LED 332 is activated by a first LED driver 340, the second LED 334 is activated by a second LED driver 342, and the third LED 336 is activated by a third LED driver 344.

The laser diode 330 may be an infrared diode that emits light having a wavelength in the range of about 805 nm to about 810 nm. The laser diode 330 may emit light having a wavelength of about 808 nm. Preferably, the first LED 332 emits light in the blue wavelength spectrum, the second LED 334 emits light in the green wavelength spectrum, and the third LED 336 emits light in the red wavelength spectrum.

A first dichroic filter 350 may be positioned in front of the laser diode 330, a second dichroic filter 352 may be positioned in front of the first LED 332, and a third dichroic filter 354 may be positioned in front of both the second LED 334 and the third LED 336. The dichroic filters 350, 352, 354 are each designed to reflect certain wavebands of light and allow passage of other wavebands of light. The first dichroic filter 350 allows the light from all three LEDs 332, 334, and 336 (e.g., light in the blue, green, and red wavelength spectra) to pass, while reflecting the laser light from the laser diode 330. The second dichroic filter 352 allows light from the second and third LEDs 334, 336 to pass while reflecting light from the first LED 332. The third dichroic filter 354 allows light from the third LED 336 to pass, while reflecting light from the second LED 334.

A first optical lens 366 may be located between the first dichroic filter 350 and the second dichroic filter 352 for focusing light received from the second dichroic filter 352. A second optical lens 368 is located between the second dichroic filter 352 and the third dichroic filter 354 for focusing light received from the third dichroic filter 354. A third optical lens 370 may be provided for focusing light received from the first dichroic filter 350. A fiber optic cable 380 may be connected to the illuminator 300 to carry the light generated by the illuminator 300 to an endoscope.

A controller 364 may control activation and modulation of the illumination sources (via control of the laser diode driver 338, the first LED driver 340, the second LED driver 342, and the third LED driver 344) according to various modes and control signals that may be received from, for example, camera control unit 112 and/or image processing unit 116 via control input 310. An exemplary illumination mode for visible light imaging can include a visible white light mode in which the laser diode 330 is off, the first LED 332 is on, the second LED 334 is on, and the third LED 336 is on. An exemplary illumination mode for fluorescence imaging can include the laser diode 330 on and the LEDs 332, 334, and 336 off. An exemplary illumination mode for combined visible light and fluorescence imaging can include green and blue LEDs on (e.g., LED 332 and LED 334 on) and the laser diode and red LED being alternately pulsed. An alternative exemplary illumination mode for combined visible light and fluorescence imaging can include green, blue, and red LEDs (e.g., LED 332, LED 334, and LED 336) pulsed in concert with pulsing of the laser diode such that the LEDs are off when the laser diode is on.

As discussed further below, illuminator 300 may be configured to switch from operating in one or more illumination modes for imaging tissue in a surgical cavity to one or more defogging modes in which the level of at least a portion of the illumination light provided during the one or more illumination modes is increased to heat the endoscope, particularly the distal end of the endoscope and/or the proximal end of the endoscope to clear fogging from one or more optical components of the endoscope and/or to prevent fogging from occurring.

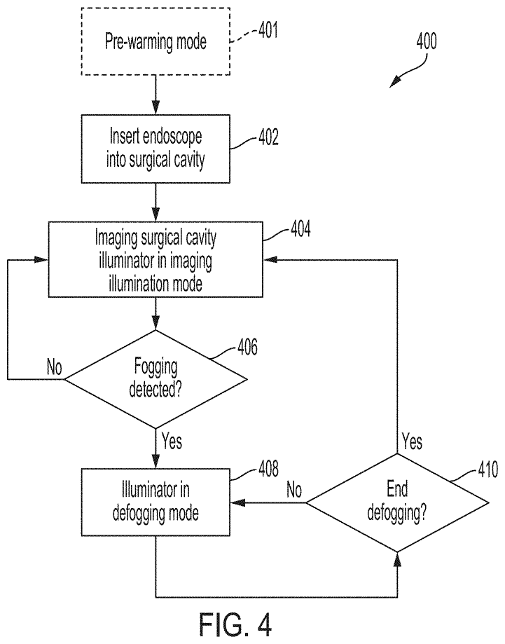

FIG. 4 illustrates a method 400 for heating a distal end of an endoscope for defogging or preventing fogging of one or more optical components at the distal end of the endoscope. Method 400 can be performed by an imaging system, such as system 100 of FIG. 1, that includes an illuminator generating illumination light and an endoscopic camera having an endoscope.

In this example, prior to the start of the defogging method 400, at step 401 the endoscope can be pre-warmed. Also prior to the defogging method 400, at step 402, the endoscope is inserted into the surgical cavity. The defogging method 400 starts at step 404, wherein the endoscopic camera captures images of the surgical cavity based on illumination light provided by the illuminator, such as illuminator 120 of FIG. 1 or illuminator 300 of FIG. 3, and transmitted to the surgical cavity by the endoscope. The illuminator operates in an imaging illumination mode in which light of a spectrum and intensity suitable for endoscopic imaging, according to one or more imaging modalities, is provided to the endoscope and directed by the endoscope to the tissue in the surgical cavity. The illuminator may operate in the imaging illumination mode based on control from one or more external components, such as camera control unit 112 or image processing unit 116 of system 100. Light reflected by the tissue, or emitted by the tissue in the case of fluorescence, is detected by the imaging sensor of the endoscopic camera and one or more images are generated based on the detected light. Images captured during step 404 (e.g., individual images or video) may be displayed on a display.

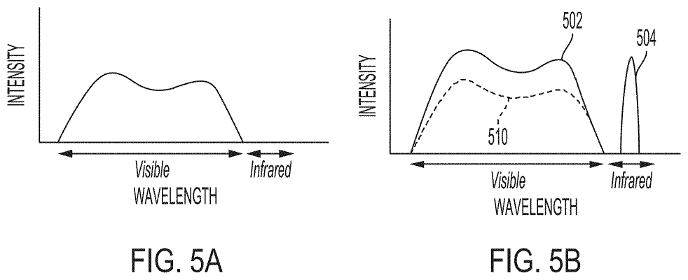

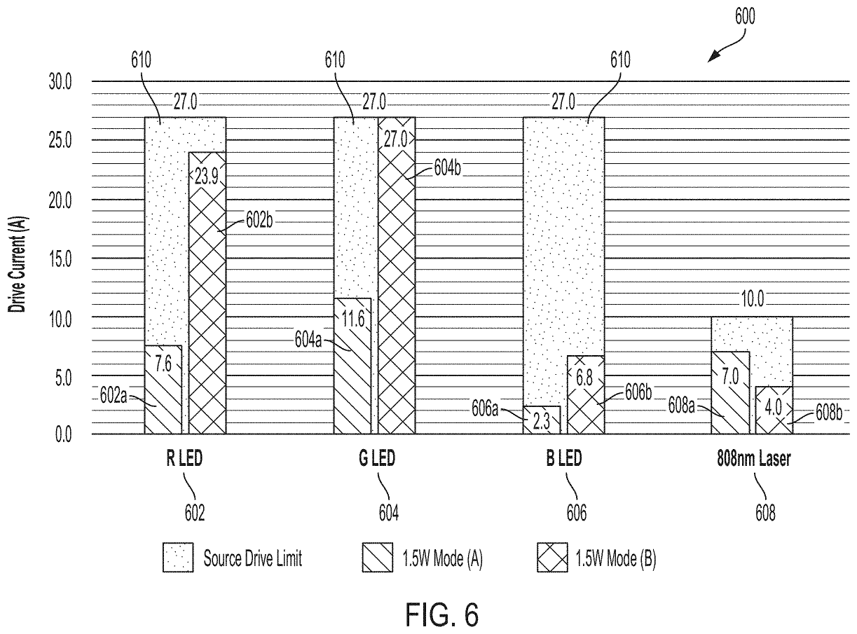

While the illuminator is operating in one or more imaging illumination modes, one or more light sources of the illuminator are activated and operating at one or more power levels suitable for endoscopic imaging. FIG. 5A illustrates an example of a light spectrum generated by an illuminator, such as illuminator 120 of FIG. 1 or illuminator 300 of FIG. 3, operating in a white light imaging mode in which one or more illumination sources are operated for generating white light at an intensity suitable for white light imaging by the endoscopic camera. The power levels of one or more light sources may be adjusted during the imaging illumination mode, such as to increase or decrease brightness and/or to adjust a color balance. The illuminator may change from one illumination mode to another illumination mode by activating or deactivating one or more light sources and/or changing the power level of one or more light sources. Optionally, one or more light sources are activated and deactivated periodically during a single illumination mode. For example, in a combined visible light and fluorescence light imaging mode, a laser diode light source producing fluorescence excitation light and a red light source may be alternately pulsed for alternately capturing visible light frames and fluorescence light frames. As used herein, a light source being activated during an illumination mode or a defogging mode encompasses pulsing the light source, which can be for capturing frames of different light and/or for modulating the brightness of a light source. Optionally, a power level of a light source can be increased/decreased by increasing/decreasing a drive current of the light source and/or by increasing/decreasing a pulse rate of the light source (which includes switching from pulsing to constantly on).

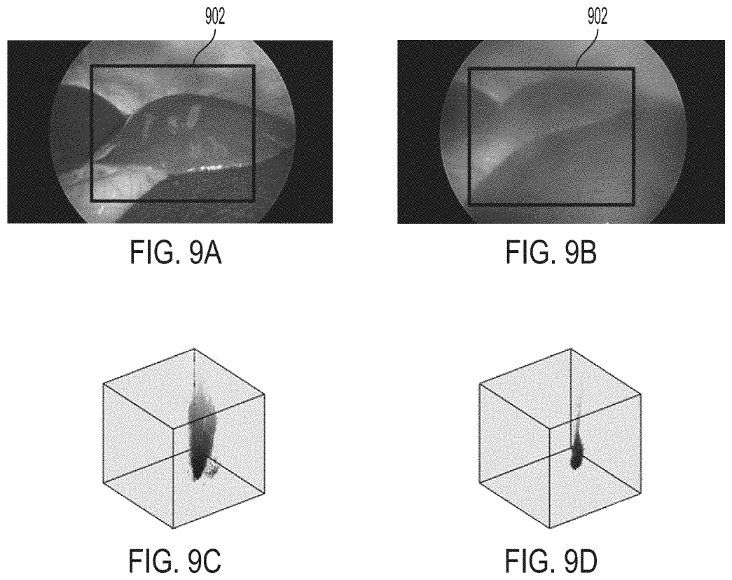

The illuminator may continue to operate in one or more imaging illumination modes (the illuminator may switch between different imaging illumination modes, such as between a white light illumination mode, a fluorescence excitation illumination mode, or a combined white light and fluorescence excitation illumination mode) until fogging of one or more optical components of the endoscope is detected at step 406. Fogging may be caused by the relatively low temperature of the endoscope as it is placed inside the surgical cavity, which brings the moisture in the surgical cavity surrounding the endoscope to its dew point, causing accumulation of condensation on the inserted portion of the endoscope, particularly one or more optical components at the distal end of the endoscope, such as the distal window or lens. As such, fogging may be detected when the endoscope has been inserted into the surgical cavity. Fogging of one or more optical components of the endoscope may also, or alternatively, occur during an ongoing procedure due to changes in the surgical cavity environment, such as due to cauterization of tissue that produces alterations in heat and moisture that may result in fogging of the endoscope. Generally, fogging is detected based on distortion in images generated by the endoscopic camera. Fogging may be detected manually by a medical personnel viewing images on a display. Fogging may be automatically detected via image processing, as discussed further below in relation to FIG. 8.

If fogging is detected at step 406, then the illuminator switches from the imaging illumination mode to a defogging mode. Optionally, the user causes the illuminator to switch modes via a user input to one or more system components. For example, a user may press a button on the endoscopic camera head that instructs the system to enter the defogging mode. The user input may be detected by, for example, the camera control unit 112 of system 100 and the camera control unit 112 may respond by sending a signal to the illuminator 120 to enter the defogging mode, or the user input may be processed by the image processing unit 116, which may send a command to the illuminator 120 to enter the defogging mode. A user input may also be an input to another user interface of the system, such as a touchscreen, keypad, switch, or other user interface on the illuminator or other system component, a voice command, or any other suitable user interface.

A command signal to the illuminator may instruct the illuminator to switch to a defogging mode and the illuminator may then operate in the defogging mode based on defogging mode operation information stored in the illuminator (for example, specifying the intensities of light from the light sources of the illuminator). In other variations, the illuminator is provided with commands for how to operate each light source to achieve the defogging illumination level. For example, the camera control unit 112 or the image processing unit 116 may transmit instructions for light source power or intensity levels for each light source that are suitable for the defogging mode.

In variations in which the system automatically detects fogging via image processing, the illuminator may be automatically instructed to switch to the defogging mode. For example, the camera control unit or image processing unit may send a command signal to the illuminator to switch to the defogging mode when fogging (or a threshold amount of fogging) has been detected. User confirmation may be required prior to commanding the illuminator to switch to the defogging mode. For example, the camera control unit or image processing unit may automatically detect fogging and may provide a prompt to the user for user confirmation that the illuminator should switch to a defogging mode. In response to receiving confirmation from the user, via any suitable user input, the camera control unit or image processing unit may send the mode switch command to the illuminator.

At step 408, the illuminator operates in the defogging mode. In switching from the imaging illumination mode to the defogging mode, the illuminator may activate one or more light sources and/or may increase the power of one or more light sources relative to that provided in the imaging illumination mode to increase the level of intensity of light provided by the illuminator to the endoscope. FIG. 5B illustrates an example of light spectra generated by the illuminator operating in a defogging mode. The defogging mode spectra are illustrated by lines 502 and 504. The imaging illumination mode spectrum of FIG. 5A is represented by dashed line 510 to illustrate that the intensity of light provided in the defogging mode in the illustrated example has increased relative to that provided in the illumination mode. As illustrated, the intensity of light has been increased across the spectrum provided in the imaging illumination mode, and, additionally, an infrared light source has been activated to provide illumination light in the infrared waveband, as shown by line 504.

The additional light energy provided during the defogging mode may manifest primarily as additional illumination into the surgical cavity but also results in additional heat loss at the distal end of the endoscope and/or additional heat loss at the light post in the proximal region of the endoscope. The heat loss warms the distal end of the endoscope, including one or more optical components at the distal end of the endoscope, such as a distal window or lens. The heat loss may similarly warm the proximal region of the endoscope, including one or more optical components at the proximal end of the endoscope, such as a proximal window or lens. The amount of additional illumination intensity provided by the illuminator during the defogging mode may be set so that the temperature of the distal window of the endoscope will reach equal to or greater than the temperature within the surgical cavity, resulting in the clearing of fog present on the exposed surface of the distal window or lens of the endoscope.

The increase in illumination light provided by the illuminator during the defogging mode can be achieved using any combination of light source activation and light source drive power. For example, the level of illumination light can be increased by increasing the power level of one or more light sources relative to the power level used during imaging illumination, by activating additional light sources, or by a combination of these two. The light sources of an illuminator may be driven in the defogging mode to achieve a target optical power (the energy of light per unit time) at the output of the endoscope and the combination of light source activations and drive power can be selected to achieve the target optical power. An illuminator may be configured to operate in a plurality of defogging modes in which the defogging mode used for a given situation can depend on, for example, the type of endoscope being defogged.

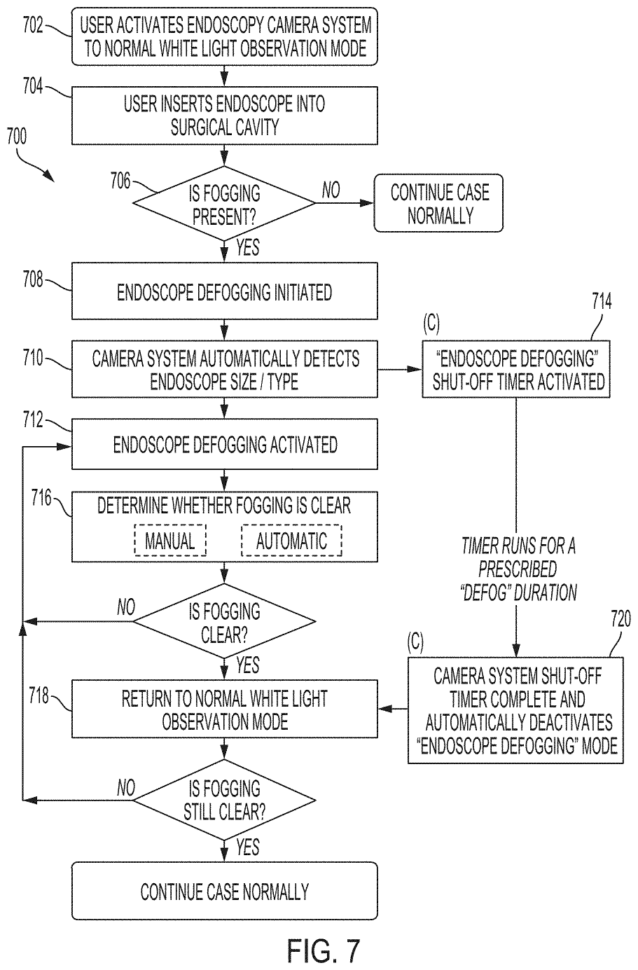

FIG. 6 is a bar graph of illuminator light source drive currents illustrating two exemplary illuminator defogging modes. The defogging modes can be provided by an illuminator such as illuminator 300 of FIG. 3. Bar graphs providing the drive current for four light sources are included—a red LED 602, a green LED 604, a blue LED 606, and an infrared laser 608. In an imaging illumination mode, output from the red, green, and blue LED light sources can be combined to provide white light and the infrared laser can be used for providing fluorescence excitation light during one or more imaging illumination modes. The bar graph 600 includes a maximum drive current limit 610 for which each light source is rated, which represents one possible limiting factor for the maximum amount of light provided by each light source. However, other limits may be imposed on the drive power of a light source, including hardware and software limits, such as safety limits associated with the power draw of the illuminator and/or with the light intensity provided by the illuminator.

A first exemplary defogging mode is illustrated by bars 602a, 604a, 606a, and 608a. In this mode, the red, green, and blue LEDs 602, 604, and 606 are driven to provide the same amount of illumination light provided in a white light illumination mode at the brightest setting and additional light energy for defogging is provided by also activating the infrared laser 608, which in the illustrated example is driven at seventy percent of the maximum rated drive current. This defogging mode maintains the color balance and intensity of the visible light sources from the imaging illumination mode so that the user can continue visible light imaging while the illuminator is in the defogging mode, which can be useful for continuing the medical procedure when, for example, the fogging does not completely obscure the field of view.

A second exemplary defogging mode is illustrated by bars 602b, 604b, 606b, and 608b. In this defogging mode, the red, green, and blue LEDs are driven at higher levels relative to those of the first defogging mode. The infrared laser 608 is also activated in this mode but at a lower drive current than in the first defogging mode. The second defogging mode relies on less infrared light energy than the first defogging mode, which may be important for patient safety.

According to various aspects, both the first and second illumination modes in FIG. 6 can provide the same optical power-1.5 Watts in this example. Thus, the examples of FIG. 6 illustrate that a given optical power, which corresponds to the amount of heating at the endoscope, can be achieved using different combinations of light source activations and drive power.

The illuminator may remain in the defogging mode at least until the one or more optical components of the endoscope that are fogged reach a temperature sufficient to clear the fogging. The amount of time required to clear the fogging will generally depend on the amount of light energy provided to the endoscope while the illuminator is in the defogging mode, environmental factors such as temperature of the endoscope, temperature surrounding the endoscope in the surgical cavity, and relative humidity in the surgical cavity, and characteristics of the endoscope, such as size and light transmission efficiency. The defogging mode may be configured to heat the distal end of an endoscope taken from typical operating room conditions (e.g., 22 deg. C, 30% relative humidity) and placed into typical surgical cavity environment (e.g., 37 deg. C, 95% relative humidity), to clear fogging of a distal window of an endoscope within 2 minutes, preferably within 1.75 minutes, more preferably within 1.5 minutes, within 1.25 minutes, within 1 minute, within 45 seconds, or within 30 seconds. The level of illumination light provided may depend on the type of endoscope such that different types of endoscopes defog within these same timeframes. For example, the defogging time for a 10 mm, 0 degree endoscope and the defogging time for a 5 mm, 0 degree endoscope in the above conditions may both be within the same ranges above by using different illumination levels for each endoscope type.

The defogging mode may be configured to heat an optical component other than the distal window of an endoscope, additionally or alternatively to heating of the distal window. For example, in arthroscopic applications as described below, the proximal window or eyepiece at the proximal end of the endoscope may become fogged and any of the systems and methods described herein with respect to defogging of the distal window may additionally or alternatively be similarly used for defogging of the proximal window. For example, increasing or modifying the illumination light passing through the light post, such as light post 126 of endoscope 102, in the proximal region of the endoscope may increase heating of the proximal region of the endoscope from heat losses at the light post illumination light transmission interface between the endoscope and the light guide, such as light guide 136 of system 100, and the heat losses may be conducted through the proximal region of the endoscope and heat the proximal window to defog the proximal window. The diameter of an illumination optical fiber bundle in the endoscope may be smaller than the diameter of an optical fiber bundle in the light guide which may increase the heat loss in the light post at the interface between the fiber bundles of different diameter. A light cone may be used in the light post at the interface between optical fiber bundles which may increase the heat loss in the light post.

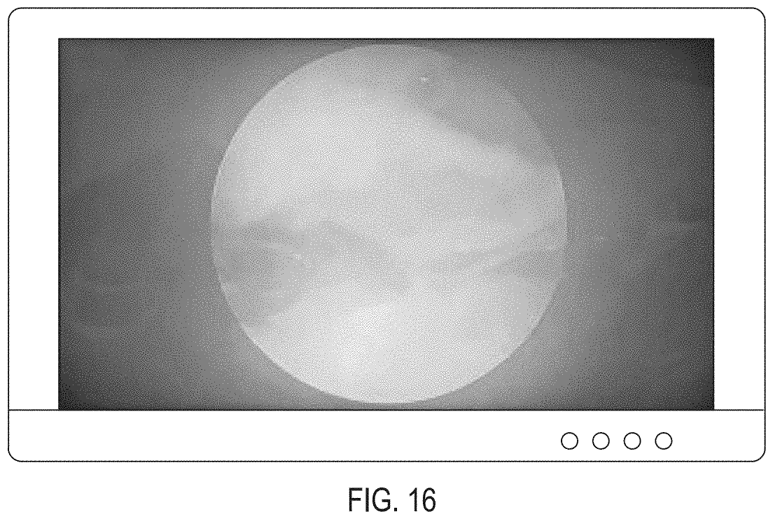

Arthroscopic surgery commonly involves filling a patent's joint with saline to expand the joint, thereby allowing the surgeon to access, visualize, and manipulate instrumentation for endoscopic surgery. During such arthroscopic surgery, fogging of the proximal window or eyepiece of the endoscope may occur, which may manifest as shown in FIG. 16 as a haze covering the entire display screen, as opposed to fogging on the distal window of an endoscope which may cause a haze covering only the scope viewing circle. Fogging of the proximal window of the endoscope during arthroscopy can occur for several reasons, including: the procedure and camera equipment can be very wet due to saline commonly leaking out of the joint through access ports, saline being able to leak from interconnections of the arthroscopic instrumentation, and saline being present on the surgeon's hands due to manipulation of arthroscopic instrumentation or patient anatomy; and arthroscopic surgery sites are located in joint spaces which are relatively small in volume and comprising tissue that is relatively reflective, resulting in relatively lower imaging illumination optical power needed compared with other endoscopes such as laparoscopes and correspondingly less heating of the endoscope components.

Returning to FIG. 4, at step 410, the decision is made whether to end the defogging process by returning to an imaging illumination mode of step 404. The illuminator may remain in the defogging mode until a user provides a command to end the defogging mode. The illuminator may end the defogging mode after a predetermined period of time. The illuminator may end the defogging mode and return to the imaging illumination mode upon the earlier of the end of a predetermined period of time and receipt of a user command to end the defogging mode. Alternatively, the defogging mode may continue until no more fogging is detected via image analysis, as discussed further below.

The illuminator may return to the imaging illumination mode that the illuminator was in prior to switching to the defogging illumination mode. For example, the illuminator may return to a visible light imaging illumination mode at the same brightness level provided prior to switching to the defogging mode. Optionally, the illuminator switches to a default imaging illumination mode upon completion of the defogging mode.

The process returns to step 404 with continued imaging of the surgical cavity. The defogging mode can be reinitiated upon any future detection of fogging of the endoscope.

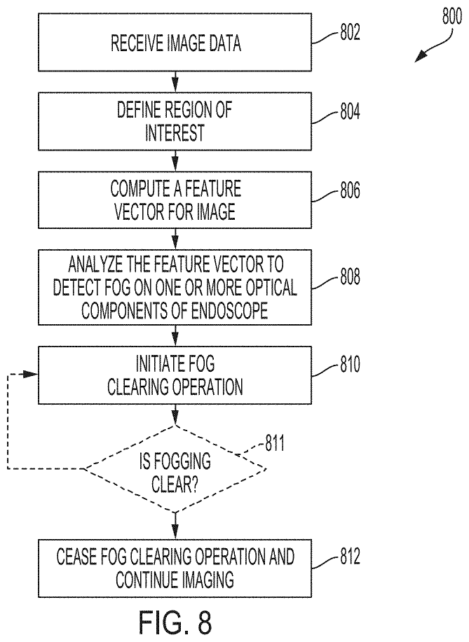

FIG. 7 is a flow diagram of a method 700 for using an endoscopic system with endoscope defogging capability, according to some variations of method 400. Method 700 begins at step 702 with the user activating an endoscopic camera system, such as system 100 of FIG. 1, in an imaging illumination mode, such as a visible light imaging illumination mode. This can include conducting a color balance calibration procedure in which the color output of the illuminator is adjusted to achieve a calibrated illumination color, as is known in the art. At step 704, the user inserts the endoscope into the surgical cavity. After the endoscope has been inserted in the surgical cavity the defogging method may start. At step 706, fogging of one or more optical components of the endoscope may be detected. As discussed above, fogging may be detected manually via the user observing fogging in the images generated and displayed by the imaging system, automatically via image processing by the imaging system, or via a combination of automatic detection and user confirmation.

If fogging is detected at step 706, the imaging system may initiate an endoscope defogging mode at step 708. The endoscope defogging mode may be initiated automatically in response to the imaging system detecting fogging in one or more images, may be initiated manually via a user input (such as a button press on the camera head, a voice command, or any other suitable user input), or may be initiated via a combination of automatic detection and user input, such as an auto-generated prompt for the user to confirm that the system should enter the defogging mode.

At step 710, the endoscopic imaging system may automatically detect a type of the endoscope. Different types of endoscopes heat differently from optical power due to one or more attributes such as size and optical transmission efficiency. The amount of optical power provided by the illuminator while in the defogging mode can be tailored to the type of the endoscope being used. For example, the amount of optical power provided when a 10 mm endoscope is being used may be greater than the optical power provided when a 5 mm endoscope is being used. The endoscopic imaging system may automatically detect the presence/absence of an endoscope connected to the system, and the system may disable the defogging mode while an endoscope is not detected to be present (i.e., if the absence of an endoscope is detected).

The endoscopic imaging system may detect the presence/absence and/or the type of endoscope in any suitable manner. The imaging system may detect a size of the endoscope by determining the size of the field-of-view portion of the images generated using the endoscope. Endoscopic images typically include a circular field-of-view portion surrounded by black. The relative size of the field-of-view portion of the image can be detected via image processing, and the detected size can be compared to stored parameters to determine scope size. The detection of a circular field-of-view portion surrounded by black may also be used to indicate the presence of an endoscope connected to the imaging system. Information associated with endoscope type may be stored in the endoscope or in the endoscopic camera head, endoscopic camera cable, or light guide cable and the information is accessed via a communication link with the camera control unit, image processing unit, or illuminator. Successful access by the endoscopic imaging system of information stored in the endoscope may be used to indicate the presence of an endoscope connected to the imaging system.

At step 712, the illuminator enters a defogging mode in which the intensity of light provided by one or more light sources of the illuminator increases to provide increased optical power to the endoscope to heat the endoscope, as discussed in detail above. Optionally, a shut-off timer can be activated at step 714. The shut-off timer can provide a maximum duration time for the illuminator to be in the defogging mode, which can help safeguard patient safety by ensuring that tissue in the surgical cavity is not exposed to high levels of illumination for an extended period.

While the illuminator is in the defogging mode, the temperature of the fogged optical component of the endoscope increases to the point that fogging clears. A determination may be made at step 716 that the fogging has cleared. This can be a manual determination made by a user via observation of one or more displayed images or an automatic determination made by the imaging system via one or more image processing techniques, as discussed further herein. If the fogging has cleared or cleared sufficiently, the illuminator may switch from the defogging mode to the imaging illumination mode at step 718. The illuminator may switch to the imaging illumination mode in response to a user command to end the defogging mode. An image processing portion of the imaging system, such as image processing unit 116 of system 100, may automatically determine that fogging has cleared via imaging processing and, in response, sends a command to the illuminator to return to the imaging illumination mode.

Optionally, the defogging mode may end prior to the fogging being cleared in the event that the shut-off timer activated in step 714 expires at step 720. The system may return to the defogging mode any time fogging is detected. However, in some variations, the illuminator does not return to the defogging mode until a predetermined “cool-down” period has elapsed since the end of the previous defogging mode operation to ensure tissue is not overexposed to high levels of illumination.

Returning to FIG. 4, the endoscope may be pre-warmed in optional step 401 to raise the temperature of the endoscope so that the endoscope does not fog when inserted into the surgical cavity in step 402. Pre-warming may be achieved using a defogging mode of the illuminator. Optionally, the user instructs pre-warming of the endoscope via one or more user inputs. A predetermined pre-warming defogging mode can be used. This pre-warming defogging mode may provide a higher level of illumination than would typically be used during imaging, which may be acceptable due to the endoscope being outside of the surgical cavity and, therefore, posing less danger to the patient.

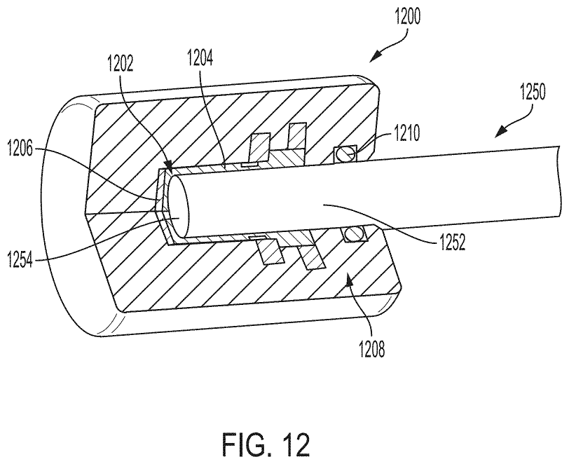

Optionally, a pre-warming cap may be used in combination with endoscope illumination to heat the endoscope prior to insertion into the surgical cavity. FIG. 12 illustrates an example of a warming cap 1200 for enhancing warming of a distal end of an endoscope using the light transmitted by the endoscope. Cap 1200 is positioned over the distal end of the endoscope and illumination light is provided to the endoscope. Light output through the endoscope light fibers is directed onto the inner layer material of the warming cap, resulting in heat generation in the form of both radiance (reflection from the inner layer material back to the distal window) and conduction (heat transferred from the inner layer material to around the endoscope outer tube).

Light provided to the endoscope when using the pre-warming cap 1200 can be any suitable wavelength band, including visible wavelengths, infrared wavelengths, ultraviolet wavelengths, or any combination thereof. In some variations, the intensity of light provided is an intensity that may typically be used during imaging, while in other variations, a higher level of intensity of light is provided to reduce the amount of time required for heating the distal end of the endoscope.

Optionally, multiple warming levels may be used to provide both fast heating and maintenance of the endoscope temperature. For example, upon initial start with a cold endoscope, the endoscope warming mode may provide a high intensity level, where a relatively high amount of illumination energy is delivered to the warming cap to rapidly heat the endoscope. Later, once the distal portion of the endoscope is sufficiently heated, the endoscope warming mode change to a maintenance intensity level, in which a medium or low amount of energy is delivered to the warming cap to maintain the warm temperature of the distal portion of the endoscope.

Cap 1200 may include a sleeve 1202 for removable positioning on the distal portion 1252 of the endoscope 1250. The sleeve 1202 may include a bore 1204 for sliding over the distal portion 1252 of the endoscope 1250 and a closed end portion 1206 that is adjacent an optical component 1254 of the endoscope 1250 when the cap 1200 is mounted on the endoscope 1250. The closed end portion 1206 may be configured to reflect at least a portion of light emitted from the distal portion 1252 of the endoscope 1250 onto the distal portion 1252 of the endoscope 1250 to heat the distal portion 1252 of the endoscope 1250. The sleeve 1202 may be configured so that the endoscope 1250 can be inserted into the sleeve 1202 until the endoscope 1250 bottoms out of the closed end portion 1206 when fully inserted into the sleeve 1202.

The cap 1200 may include an insulating portion 1208 that at least partially surrounds the sleeve 1202 for thermally insulating the sleeve 1202. The sleeve 1202 and the insulating portion 1208 may be made from the same material or may be made from different materials. The sleeve 1202 may be formed of a metal and the insulating portion 1208 us formed of a polymeric material.

A resilient retainer 1210 may be positioned in the insulating portion 1208 or in the sleeve 1202 for resiliently positioning against an outer surface of the distal portion 1252 of the endoscope 1250 to retain the cap 1200 on the endoscope 1250 and to prevent light from escaping the cap 1200. Optionally, the resilient retainer 1210 is an elastomeric o-ring.

Cap 1200 may be a passive unit that does not use any electrical power. Heating of the endoscope may be achieved via the light transmitted through the endoscope.

The cap 1200 may be a disposable cap that is intended to be used for a single imaging session and then discarded. Alternatively, the cap 1200 is reusable and configured to be sterilized as a unit (i.e., without requiring disassembly).

The cap 1200 may be configured differently for different types of endoscopes. For example, a first cap size may be configured with a larger bore for a 10mm endoscope and a second cap size may be configured with a smaller bore for a 5mm endoscope.

The cap may be used by mounting the cap 1200 on the distal portion 1252 of the endoscope 1250 prior to inserting the endoscope 1250 into the surgical cavity. Then, light is provided from an illuminator, such as illuminator 120 of system 100, to the endoscope 1250 so that the light is emitted from the distal end of the endoscope 1250. The illuminator may operate in a normal imaging illumination mode while the endoscope 1250 is warming with the cap 1200 mounted. Alternatively, the illuminator operates in a defogging mode in which an increased level of illumination light is provided, as discussed above. The distal portion 1252 of the endoscope 1250 may warm based solely on the light provided from the illuminator to the endoscope 1250. The warming of the endoscope 1250 using the cap 1200 may continue for a predetermined period of time selected so that the endoscope 1250 reaches a temperature suitable for preventing fogging of the endoscope 1250 upon insertion of the endoscope 1250 into the surgical cavity. Preferably, the endoscope is warmed until reaching at least 34 degrees C. and more preferably at least 37 degrees C. Optionally, the endoscope 1250 is warmed from a typical operating room temperature of about 20 degrees C. to 37 degrees C. in within 2 minutes, preferably within 90 seconds, more preferably within 80 seconds, or within 60 seconds.

As noted with respect to methods 400 and 700, fogging of one or more optical component of the endoscope can be detected via image processing. FIG. 8 is a flow diagram of a method 800 for automatically detecting fogging on one or more optical components of an endoscope via image processing. Method 800 can be performed by one or more image processors (for example, a configured FPGA or a CPU running one or more software programs) of an endoscopic imaging system, such as one or more processors of camera control unit 112 or image processing unit 116 of system 100 of FIG. 1. Although method 800 is described below as being used for detecting fogging on one or more optical components of an endoscope, method 800 can be used for detecting other deposits on the optical components of the endoscope. For example, method 800 can be used to detect smudging on the distal window of the endoscope.

An image processor employing method 800 may detect fogging and/or other deposits on one or more optical components of the endoscope by calculating a feature vector based on one or more features associated with one or more images and using a machine learned model to determine whether the determined feature(s) are indicative of deposits, such as fog, on the one or more optical components that obscure the clarity of the field of view. A feature vector may be computed for an image via sliding window computer vision technique and the feature vector is input to a single class classifier, such as Support Vector Machine classifier for a single class, which generates a prediction as to whether the feature vector indicates fogging and/or other deposits on the one or more optical component.

At step 802, image data is received at an image processor. The image data may be received from an endoscopic camera, such as endoscopic camera head 108 of system 100, or from camera control unit 112 of system 100. The image data corresponds to light received at one or more imaging sensors from a field of view of an endoscope. The image data can include one or more snapshot images and/or one or more video frames.

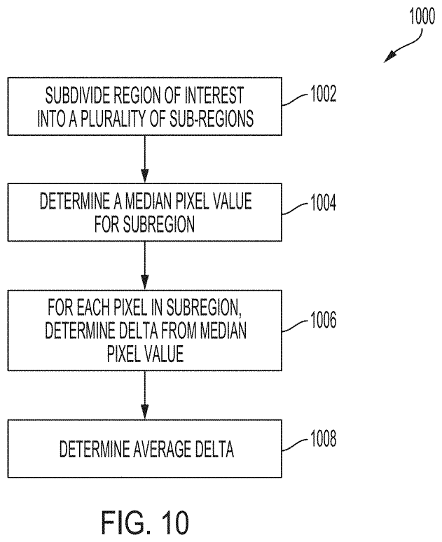

At step 804, at least one region of interest of at least one image (single snapshot image or video frame) from the image data is defined. The region of interest can be the entire image or can be any suitable portion of the image. A plurality of regions of interest may be defined for an image. Regions of interest may be overlapping or non-overlapping. A region of interest can be any suitable size and shape. An exemplary region of interest 902 is illustrated in FIG. 9A and 9B, which illustrate a non-foggy image and a foggy image, respectively.

At step 806, at least one feature vector is computed for the region(s) of interest defined in step 804. A feature vector includes one or more numeric values associated with one or more features of the region of interest that correlate with foggy versus non-foggy images. A feature vector can include any number of features, including just a single feature. Suitable features may be those that measure the variability in pixel values across the window. Since fogginess on an optical component disperses light, a foggy image will tend to have less variability in pixel values within a window. This is illustrated in FIGS. 9C and 9D, which show the variability of pixel values for the non-foggy image of a scene shown in FIG. 9A and the foggy image of the scene shown in FIG. 9B. FIG. 9C is a plot of the pixel values in RGB space for the foggy image of FIG. 9A and FIG. 9D is a plot of the pixel values in RGB space for the non-foggy image of FIG. 9B. As is apparent in comparing the two plots, the pixel values for the foggy image (FIG. 9C) are more clustered than the pixel values for the non-foggy image.

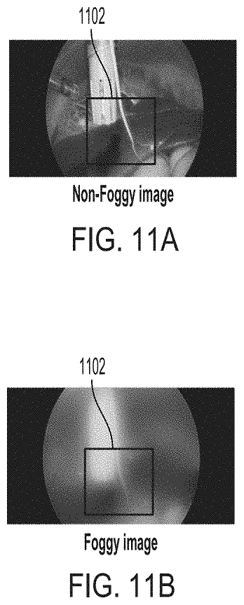

Various features and combinations of features can be used to generate the feature vector. A specific feature that has been discovered by the inventors to be particularly suitable for fog detection in endoscopic imaging is dispersion index. FIG. 10 illustrates an exemplary method 1000 for calculating dispersion index.

At step 1002, an image is subdivided into a plurality of sub-regions. The sub-regions can be any suitable size and shape. For example, sub-regions may be 20×20, 50×50 pixels, or 100×100 pixels. The sub-regions can be overlapping or non-overlapping. The sub-regions can all be the same size or can have different sizes. For example, a first set of sub-regions can be sets of 50×50 pixels that cover the region of interest and a second set of windows can be sets of 100×100 pixel that cover the same region of interest. The region of interest may be subdivided using a sliding window technique.

At step 1004, for each sub-region defined in step 1002, a median pixel value for the respective sub-region is determined. Then, at step 1006, each pixel value in the respective sub-regions is compared to the median pixel value and a difference is calculated. Next, at step 1008, the average of pixel value differences from the median pixel value is determined. This average is the dispersion index for the respective sub-region.

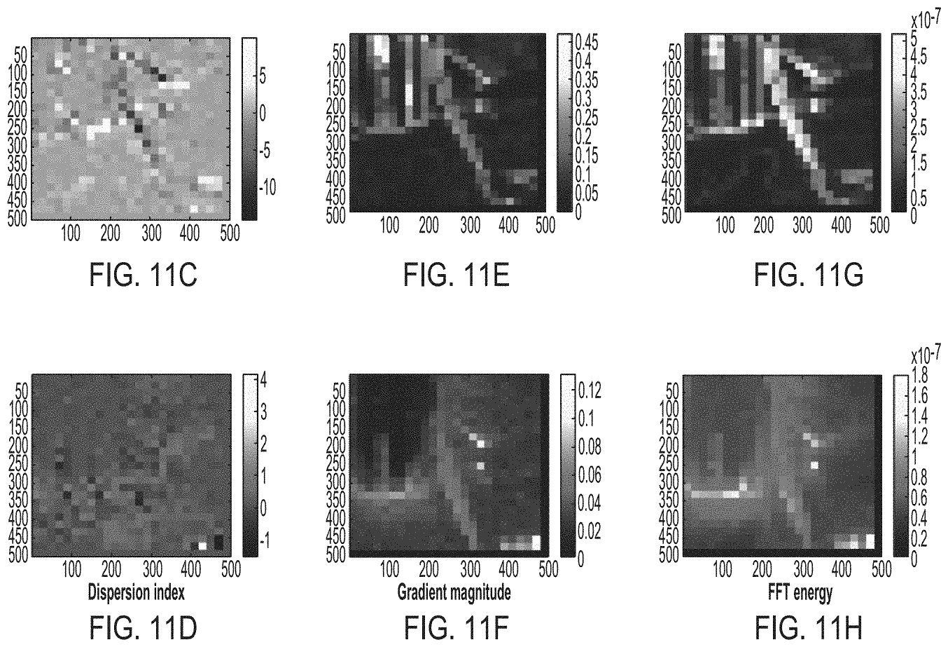

FIGS. 11C and 11D illustrate the results of a dispersion index calculation for region of interest 1102 in the non-foggy image of FIG. 11A and the foggy image of FIG. 11B. For each of the foggy and non-foggy images, region of interest 1102 has been subdivided into sub-regions of 20×20 pixels and the dispersion index has been calculated. The heat maps of FIG. 11C and 11D provide the dispersion index for each sub-region. The scale for the non-foggy image heat map of FIG. 11C is −15 to 10 and the scale for the foggy image heat maps of FIG. 11D is −2 to 4. Comparing FIG. 11C and 11D, it is apparent that range of the dispersion index values for the non-foggy image is larger than the range of those for the foggy image, indicating less pixel value variation in the foggy image due to the dispersion of light caused by the fogging of an optical component of the endoscope.

Other examples of features that can be used in step 806 of method 800 include gradient magnitude and FFT (Fast Fourier Transform) energy. Although these techniques are known in the art of image processing, the inventors discovered that these features are unexpectedly particularly suited for detecting fogginess in an image. FIG. 11E and 11F are heat maps of the gradient magnitude computed for the 20×20 sub-regions for the region of interest 1102 in the non-foggy image of FIG. 11A and the foggy image of FIG. 11B, respectively. FIG. 11G and 11H are heat maps of the FFT energy computed for the 20×20 sub-regions for the region of interest 1102 in the non-foggy image of FIG. 11A and the foggy image of FIG. 11B, respectively.

A feature vector for a region of interest of an image can include, for example, dispersion index, gradient magnitude, and FFT energy computed for each of a plurality of sub-regions of the region of interest. The inventors discovered that this particular combination of features unexpectedly enables reliable predictions of foggy versus non-foggy regions of interest.

Returning to FIG. 8, at step 808, the at least one feature vector computed in step 806 is analyzed to detect fogging of the at least one optical components of the endoscope. The at least one feature vector may be analyzed using a Machine Learning algorithm trained on foggy and non-foggy endoscopic images. The Machine Learning algorithm may be trained on labeled images. The Machine Learning algorithm may be a classifier. The classifier can be a single class classifier. The single class classifier may be a Support Vector Machine. The at least one feature vector may be fed to the Machine Learning algorithm and the Machine Learning algorithm classifies the at least one feature vector as corresponding to fog or no-fog. Optionally, a multi-class classifier is used to detect multiple types of deposits on the endoscope. For example, the multi-class classifier may be configured to classify regions of interest as having fog, having smudging, or being clear.

Optionally, a plurality of regions of interest are defined for an image, at least one feature vector is determined for each of the regions of interest, and a prediction regarding the presence of fog in the image is made for each of the plurality of regions of interest.

At step 810, a fog clearing operation is initiated based on fog being detected in at least one region of interest in at least one image. A decision to perform the fog clearing operation may be based on fog spread—fogging being detected repeatedly over a time series of images. A determination to perform a fog clearing operation may depend on detecting fogging in a threshold number of images in a time series of images or a predefined proportion of the time series of images. The threshold may be set according to the desired balance between continuous imaging and fog-free imaging. The lower the threshold, the sooner the fog clearing operation will be performed and the less time that the field of view will be obscured by fog, but the greater the chance of false positives (detecting fogging when none is present) and the sooner a fog clearing operation potentially prevents or hinders visualization of the field of view. Conversely, the higher the threshold, the less of a chance of false positives and the longer the delay until the defogging operation, but the greater the potential for the user to be viewing foggy images.

A decision to perform the fog clearing operation may be based on fog density in an image i.e., the presence of fog in a predefined proportion of an image. For example, an image that has fog on only thirty percent of the field of view may be insufficiently foggy to perform the fog clearing operation. Optionally, a plurality of regions of interest are defined for an image, a fog versus no-fog determination is made for each of the regions of interest, and a decision to perform a defogging operation is be based on a threshold number of the regions of interest having fog. Any suitable threshold can be used. Example thresholds include at least 20%, at least 50%, at least 75%, etc.

Optionally, a decision to perform a fog clearing operation is based on a combination of fog density and fog spread. For example, the defogging operation is only performed once a predefined number of images of a time series of images are determined to have sufficient density of fog. Requiring fog density and/or fog spread can prevent erroneous fog predictions from causing performance of a defogging operation and can prevent the defogging operation from being performed prematurely.

Upon determining that fog is present in an image or upon meeting the threshold requirements of fog spread and/or fog density, a fog clearing operation is performed. The fog clearing operation may include heating the distal end and/or proximal end of an endoscope using illumination light from an illuminator, as discussed above with respect to method 400 of FIG. 4.

Other examples of fog clearing operations are spraying a liquid or gas on the distal end of the endoscope to clear the condensation from the distal end of the endoscope. This can be done, for example, by directing fluid or gas through one or more channels provided in the wall of the endoscope or in the wall of a sheath within which the endoscope is positioned to a nozzle that directs the fluid or gas at the distal end of the endoscope. The fluid or gas may be heated, for example, by a resistive heating wire extending through the gas flow channel in the endoscope or sheath. The flow of the gas can be controlled by an insufflator that receives commands from, for example, the image processing unit 116 of system 100.

Another example of a fog clearing operation is the activation of a resistance heater located inside the endoscope near, adjacent to, or integrated within the lens of the scope. For rigid laparoscopes, the resistance heater can be powered by conductors built into the eyepiece of the scope that come into contact with mating conductors on the camera head coupler, which are powered through mating contacts in the camera head, which receives its power from the camera control unit. For flexible endoscopes, the power to the resistance heater may be conducted through wires running inside the scope and camera.

Optionally, in response to determining that a sufficient amount of fogging is present in the images generated using the endoscope, the one or more processors (for example, one or more processors of the camera control unit 112 or image processing unit 116 of system 100) provide a control command to a component for performing the defogging operation. For example, the one or more processors may generate a command to initiate a defogging operation and the command may be provided to the illuminator to perform an illumination-based defogging operation as discussed above. A command may be sent to an insufflator for initiating a defogging operation that sprays a gas or fluid on the distal end of the endoscope for defogging. Optionally, fogging is automatically detected in one or more images as discussed above but prior to the defogging operation being initiated, a user input confirming that the defogging operation should be performed may be required. This may be done in any suitable fashion, such as by providing a prompt on a graphical user interface on a display.

One of a plurality of optical component clearing operations can be selected based on a class of deposits determined by a multi-class classifier in step 808. For example, where the classifier determines that the region of interest is foggy, a defogging operation may be performed and where the classifier determines that the region of interest has smudging, a de-smudging operation may be performed.

At step 812, the defogging operation ceases and the imaging continues normally. For example, where the level of illumination light is increased to heat the distal end of the endoscope to clear the fogging, the level of illumination light returns to a level suitable for imaging and imaging continues normally. Optionally, the defogging operation lasts for a predefined period and the defogging ceases once the predefined period has elapsed. For example, the defogging operation may be configured to continue for a certain number of seconds and once that time has elapsed, the defogging operation ceases and imaging continues normally.

Optionally, one or more images generated during the defogging operation are analyzed to determine when the fogging has been cleared or sufficiently cleared at step 811. Techniques similar to those discussed above for detecting fogging can be used to detect that fogging has been sufficiently cleared from the one or more optical components of the endoscope. The defogging operation may continue until no fogging is found in a region of interest of an image. The defogging operation may continue until the density of fogging in an image and/or the spread of fogging across a time series of images drops below predefined thresholds. One or more spread or density thresholds used for determining when the defogging operation ends can be different than thresholds used for determining when the defogging operation should begin. For example, a density threshold for starting the defogging operation can be 30% or more of an image covered by fog and the density threshold for ceasing the defogging operation can be 10% or less of an image covered by fog.

Analysis of images to determine fogging and/or the sufficient clearing of fogging can be performed on each image received while operating the endoscope during a medical procedure. Image analysis may be performed on fewer than all images generated during an imaging session or portion of an imaging session. For example, every second, third, fourth, etc., image in a time series of images may be analyzed to detect defogging and/or sufficient defogging. A proportion of images of a series of images that are analyzed for detecting fogging can be inversely related to the frame rate, which can help conserve computing resources.

Thus, according to various systems and methods, fogging of an endoscope lens or window can be automatically detected and a defogging operation can be automatically performed, which can decrease the amount of time that endoscope fogging adversely affects image quality and can help reduce the cognitive load on the practitioner by automating fog-related efforts that may previously have been done manually.

The distal window or lens of an endoscope can be configured to help mitigate fogging. For example, the distal window of an endoscope, such as distal window 208 of endoscope 200 of FIG. 2, can be coated with a thermochromic layer that increases in transparency as it heats and increases in temperature in response to absorbing incident light. The incident light can be light reflected or emitted from tissue and/or illumination light traveling through the endoscope. The thermochromic layer can cause the distal lens or window of the endoscope to maintain a higher temperature during imaging, which can prevent fog formation, and/or can increase the rate of heating during an illumination-based defogging procedure. For example, when the distal end of the endoscope is at a relatively lower temperature, such as at the start of use of the endoscope, the thermochromic layer may have a relatively lower transparency and correspondingly may absorb more incident light and thereby contribute to heating of the distal end of the endoscope. Once the distal end of the endoscope reaches a sufficient temperature to prevent fogging, the thermochromic layer may have a high transparency and correspondingly may reduce or cease its absorption of incident light and reduce or cease its contribution to heating of the distal end of the endoscope.

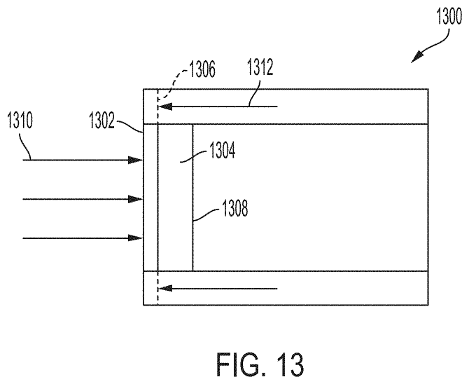

FIG. 13 illustrates a distal portion of an exemplary endoscope 1300 (which is substantially similar to endoscope 200 of FIG. 2) in which a thermochromic layer 1302 is provided on a distal window 1304 of the endoscope 1300. The thermochromic layer 1302 can be provided on the distal surface of the distal window 1304, as shown, or on the opposite, proximal surface 1308. A portion of the light 1310 from the scene interacts with the thermochromics in the thermochromic layer 1302, which convert light energy to heat. The thermochromic layer 1302 may extend over the distal ends of the plurality of fiber optics 1306 as indicating by the dashed lines to enable light 1312 exiting the fiber optics 1306 to directly interact with the thermochromic layer 1302. The thermochromic layer 1302 may be located over the distal ends of the plurality of fiber optics 1306 and does not extend over the imaging light receiving portion of the distal window 1304, with heating of the imaging light receiving portion of the distal window achieved via conduction. The thermochromic layer 1302 may be formed using thermochromic pigment mixed with an adhesive and applied in a coating to the distal window 1304 of the endoscope 1300. The thermochromic pigments can be selected to respond to any suitable bandwidth of light, including bandwidths in the visible spectrum, the infrared spectrum, and/or the ultraviolet spectrum. The thermochromic pigments may be selected to minimize distortion of imaging. For example, the thermochromic pigments may interact with a relatively small proportion of the imaging light or with portions of light that would otherwise be filtered by the endoscopic imaging system (such as fluorescence excitation light).

The distal window of the endoscope, such as distal window 208 of endoscope 200 of FIG. 2, may be laser etched with an array of nano-structures configured to retard formation of water condensation droplets on the distal window. The etched nano-structures array may be configured to that have hydrophobic properties to prevent or retard the condensing of water on the distal window. The distal window may be laser etched with a nano-structure that is based on the ‘moth eye’ effect, which is a known anti-reflection technique but has been discovered to also have hydrophobic properties. Optionally, the etched nano-structure array is configured to have hydrophilic properties that can help prevent droplet formation by encouraging condensation to disperse into an even layer, increasing the spatial uniformity of incident light rays. This can help prevent the light scattering effects caused by droplets, reducing the loss in image clarity otherwise caused by fogging.

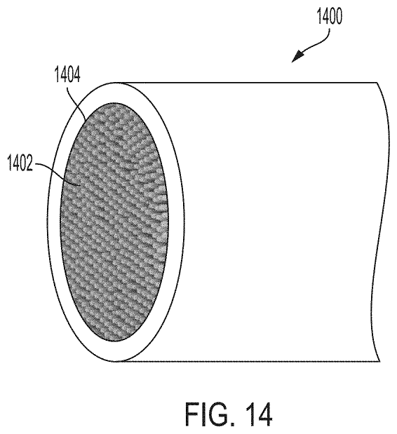

FIG. 14 is a conceptual illustration of etched nano-structure array 1402 on a distal window 1404 of a distal portion of an endoscope 1400. The distal window 1404, which can be made of sapphire, a commonly used material that is scratch-resistant due to its hardness, can be etched (e.g., chemical etching, laser etching) to form a nano-structure array 1402. The durability of the base sapphire substrate can ensure that the etched structures are durable and can withstand endoscope sterilization and other reprocessing in which the endoscope may be subjected to heat, humidity, hydrogen peroxide plasma, enzymes, chemicals, and/or abrasives. The etched nano-structure array 1402 can be configured to minimally affect transmission of light in the desired imaging wavelength band(s). Moreover, the etched nano-structure array 1402 can improve imaging via its anti-reflection properties. Although in the example of FIG. 14 the etched nano-structure array 1402 is shown on a distal window of an endoscope 1400, in other variations, the etched nano-structure array may be located on another optical component of endoscope 1400, such as for example a proximal window of endoscope 1400.

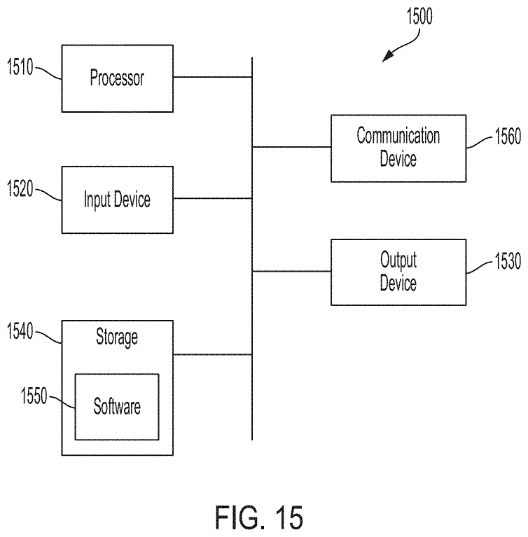

FIG. 15 illustrates an example of an exemplary computing system 1500 that can be used for one or more of components of system 100 of FIG. 1, such as one or more of camera head 108, camera control unit 112, image processing unit 116, and illuminator 120. System 1500 can be a computer connected to a network, such as one or more networks of hospital, including a local area network within a room of a medical facility and a network linking different portions of the medical facility. System 1500 can be a client or a server. As shown in FIG. 15, system 1500 can be any suitable type of processor-based system, such as a personal computer, workstation, server, handheld computing device (portable electronic device) such as a phone or tablet, or dedicated device. The system 1500 can include, for example, one or more of input device 1520, output device 1530, one or more processors 1510, storage 1540, and communication device 1560. Input device 1520 and output device 1530 can generally correspond to those described above and can either be connectable or integrated with the computer.

Input device 1520 can be any suitable device that provides input, such as a touch screen, keyboard or keypad, mouse, gesture recognition component of a virtual/augmented reality system, or voice-recognition device. Output device 1530 can be or include any suitable device that provides output, such as a display, touch screen, haptics device, virtual/augmented reality display, or speaker.

Storage 1540 can be any suitable device that provides storage, such as an electrical, magnetic, or optical memory including a RAM, cache, hard drive, removable storage disk, or other non-transitory computer readable medium. Communication device 1560 can include any suitable device capable of transmitting and receiving signals over a network, such as a network interface chip or device. The components of the computing system 1500 can be connected in any suitable manner, such as via a physical bus or wirelessly.

Processor(s) 1510 can be any suitable processor or combination of processors, including any of, or any combination of, a central processing unit (CPU), field programmable gate array (FPGA), and application-specific integrated circuit (ASIC). Software 1550, which can be stored in storage 1540 and executed by one or more processors 1510, can include, for example, the programming that embodies the functionality or portions of the functionality of the present disclosure (e.g., as embodied in the devices as described above). For example, software 1550 can include one or more programs for performing one or more of the steps of method 400, method 800, and/or method 1000.

Software 1550 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1540, that can contain or store programming for use by or in connection with an instruction execution system, apparatus, or device.

Software 1550 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport computer readable medium can include, but is not limited to, an electronic, magnetic, optical, electromagnetic, or infrared wired or wireless propagation medium.

System 1500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 1500 can implement any operating system suitable for operating on the network. Software 1550 can be written in any suitable programming language, such as C, C++, Java, or Python. Application software embodying the functionality of the present disclosure can be deployed in different configurations, such as in a client/server arrangement or through a Web browser as a Web-based application or Web service, for example.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for defogging an optical component of an endoscope, the method comprising:
operating an illuminator in an imaging mode in which the illuminator generates illumination light for endoscopic imaging of a target, wherein at least a portion of the illumination light is generated by a light source that generates light having a first waveband at an imaging intensity level; and
changing the operating mode of the illuminator from the imaging mode to a defogging mode in which an intensity level of the light having the first waveband is increased from the imaging intensity level to a defogging intensity level to warm and defog the optical component of the endoscope.

2. The method of claim 1, further comprising changing the mode of the illuminator back to the imaging mode after the optical component of the endoscope is at least partially defogged.

3. The method of claim 2, wherein the mode of the illuminator is changed back to the imaging mode after a predefined period of time in the defogging mode.

4. The method of claim 1, wherein the illuminator comprises a plurality of light sources, each light source of the plurality of light sources generates a different waveband of light, and increasing the intensity level of the illumination light having the first waveband comprises increasing power provided to at least the light source that generates the light having the first waveband from an imaging power level to a defogging power level.

5. The method of claim 4, wherein the light source that generates the light having the first waveband comprises a plurality of light emitters that each emit light having the first waveband.

6. The method of claim 1, wherein the first waveband is a waveband in the visible light spectrum.

7. The method of claim 1, wherein the first waveband is a near infrared waveband for exciting a fluorescence target.

8. The method of claim 1, wherein the first waveband is an ultraviolet waveband.

9. The method of claim 1, wherein a second portion of the illumination light is generated by a second light source generating light having a second waveband, and an intensity level of the light having the second waveband provided by the second light source is increased in the defogging mode relative to the imaging mode.

10. The method of claim 1, wherein the mode of the illuminator is changed from the imaging mode to the defogging mode in response to a user input.

11. The method of claim 1, comprising:
receiving image data generated by an endoscopic camera connected to the endoscope while the illuminator is in the imaging mode;
analyzing the image data to automatically detect fogging of the optical component of the endoscope; and
in response to automatically detecting fogging of the optical component, sending a signal to the illuminator that changes the operating mode of the illuminator from the imaging mode to the defogging mode.

12. The method of claim 11, further comprising sending a subsequent signal to the illuminator to change the operating mode of the illuminator back to the imaging mode.

13. The method of claim 11, further comprising:
receiving additional image data while the illuminator is in the defogging mode;
analyzing the image data to automatically detect that the optical component of the endoscope has been defogged; and
in response to automatically detecting that the optical component of the endoscope has been defogged, sending a signal to the illuminator to change the operating mode of the illuminator back to the imaging mode.

14. The method of claim 1, wherein the optical component comprises a distal lens or window at the distal end of the endoscope.

15. The method of claim 1, wherein the optical component comprises a proximal lens or window at the proximal end of the endoscope.

16. The method of claim 1, wherein the endoscope comprises a plurality of optical fibers for carrying the illumination light and at least a portion of the plurality of optical fibers are located radially outwardly of the optical component.

17. The method of claim 16, wherein at least a second portion of the plurality of optical fibers direct light onto the optical component.

18. The method of claim 16, wherein an outer surface of the optical component and termination surfaces of at least the portion of the plurality of optical fibers that are located radially outwardly of the optical component are coplanar.

19. The method of claim 1, wherein the illuminator is configured to operate in a plurality of defogging modes for endoscopes of different types.

20. The method of claim 1, further comprising automatically determining a type of the endoscope, wherein the defogging intensity level in the defogging mode is based at least partially on the determined type of the endoscope.

21. The method of claim 20, wherein the mode of the illuminator is changed back to the imaging mode after a predefined period of time in the defogging mode, and wherein the predefined period of time is based on the type of the endoscope.

22. The method of claim 1, further comprising automatically detecting the absence of an endoscope and, while the absence is detected, disabling the defogging mode.

23. The method of claim 1, wherein the optical component is configured to transmit the light having a first waveband.

24. The method of claim 1, wherein the endoscope is in use while the optical component of the endoscope is defogged.

25. A system comprising one or more processors configured for:
operating an illuminator in an imaging mode in which the illuminator generates illumination light for endoscopic imaging of a target with an endoscope comprising an optical component, wherein at least a portion of the illumination light is generated by a light source that generates light having a first waveband at an imaging intensity level; and
changing the operating mode of the illuminator from the imaging mode to a defogging mode in which an intensity level of the light having the first waveband is increased from the imaging intensity level to a defogging intensity level to warm and defog the optical component of the endoscope.

26. The system of claim 25, wherein the optical component comprises a proximal lens or window at the proximal end of the endoscope.

27. The system of claim 25, wherein the optical component comprises a distal lens or window at the distal end of the endoscope.

28. The system of claim 25, wherein the system is communicatively connected to the illuminator and the system is configured to send a signal to the illuminator to change the operating mode of the illuminator from the imaging mode to the defogging mode.

29. The system of claim 28, wherein the signal comprises information corresponding to the defogging intensity level.

30. The system of claim 25, wherein the one or more processors are configured for analyzing image data received from an endoscopic imager to detect fogging of the optical component and changing the operating mode of the illuminator from the imaging mode to the defogging mode in response to detecting fogging of the optical component.
